(19) United States
(12) Patent Application Publication
(10) Pub. No.: US 2024/0001177 A1
(43) Pub. Date: Jan. 4, 2024

(54) METHOD, DEVICE AND SYSTEM OF SENSOR-BASED BREATHABLE AIR QUALITY MONITORING IN A FIREFIGHTER AIR REPLENISHMENT SYSTEM

(71) Applicant: Anthony J. Turiello, Westlake, TX (US)

(72) Inventor: Anthony J. Turiello, Westlake, TX (US)

(21) Appl. No.: 18/131,869

(22) Filed: Apr. 7, 2023

Related U.S. Application Data
(60) Provisional application No. 63/356,996, filed on Jun. 29, 2022.
(60) Provisional application No. 63/357,743, filed on Jul. 1, 2022.
(60) Provisional application No. 63/357,754, filed on Jul. 1, 2022.

(51) Int. Cl. A62B 27/00; G01N 1/24; G01N 33/00; A62B 9/00

(52) U.S. Cl. CPC A62B 27/00; G01N 1/24; G01N 33/0006; G01N 33/0065; A62B 9/006

(57) ABSTRACT

Disclosed are a method, a device and a system of sensor-based breathable air quality monitoring in a safety system of a structure. In accordance therewith, an air quality analyzer is coupled to a fixed piping system installed within the structure to facilitate delivery of breathable air from a source thereof. The air quality analyzer includes an air sequestration chamber in which a portion of the breathable air is segregated for analysis as an air sample, and one or more sensor(s) to sense one or more air quality parameter(s) from the air sample within the air sequestration chamber. An analysis module of the safety system performs analysis of the sensed one or more air quality parameter(s) based on coupling thereof to the air quality analyzer and transmits data associated with the analysis over a communication link.

### CLAIM OF PRIORITY

This application is a conversion application of, and claims priority to, U.S. Provisional Patent Application No. 63/356,996 titled CLOUD-BASED FIREFIGHTING AIR REPLENISHMENT MONITORING SYSTEM. SENSORS AND METHODS filed on Jun. 29, 2022, U.S. Provisional Patent Application No. 63/357,743 titled CONTINUAL AIR QUALITY MONITORING THROUGH LOCALIZED ANALYSIS OF BREATHABLE AIR THROUGH A SENSOR ARRAY filed on Jul. 1, 2022, and U.S. Provisional Patent Application No. 63/357,754 titled ON-DEMAND CERTIFICATION THROUGH COMMUNICATION OF ASSOCIATED AIR-QUALITY MARKER DATA TO A REMOTE CERTIFICATION LABORATORY filed on Jul. 1, 2022. The contents of each of the aforementioned applications are incorporated herein by reference in entirety thereof.

### FIELD OF TECHNOLOGY

This disclosure relates generally to emergency systems and, more particularly, to a method, a device and a system of sensor-based breathable air quality monitoring in a safety system of a structure.

### BACKGROUND

A structure (e.g., a vertical building, a horizontal building, a tunnel, marine craft, a mine) may have a Firefighter Air Replenishment System (FARS) implemented therein. The FARS may be employed to provide pure and safe breathable air to emergency personnel and/or maintenance personnel associated therewith. During contamination of the breathable air through the FARS and/or anomalous levels of components (e.g., carbon monoxide, carbon dioxide) thereof, the emergency and/or the maintenance personnel may be exposed to adverse health and/or life hazards thereto.

In order to ensure that the breathable air across the FARS conforms to safety standards, an authority (e.g., a laboratory, a governmental organization) may have to test the breathable air and/or one or more components of the FARS and certify a quality of the breathable air and/or the one or more components of the FARS. Operations involved in a traditional process of certifying the FARS and/or the breathable air thereacross may prove to be tedious, time-consuming and/or ineffective in terms of efficiency and costs involved.

### SUMMARY

Disclosed are a system, a device and a method of sensor-based breathable air quality monitoring in a safety system of a structure.

In one aspect, a safety system of a structure includes a fixed piping system installed within the structure to facilitate delivery of breathable air from a source of compressed air, and an air quality analyzer coupled to the fixed piping system. The air quality analyzer includes an air sequestration chamber in which a portion of the breathable air is segregated for analysis as an air sample, and one or more sensor(s) to sense one or more air quality parameter(s) from the air sample within the air sequestration chamber. The safety system also includes an analysis module to perform analysis of the sensed one or more air quality parameter(s) based on coupling thereof to the air quality analyzer and to transmit data associated with the analysis over a communication link.

In another aspect, an air quality analyzer coupled to a fixed piping system implemented within a safety system of a structure, with the fixed piping system distributing breathable air from a source across the safety system, is disclosed. The air quality analyzer includes an air sequestration chamber in which a portion of the breathable air is segregated for analysis as an air sample, and one or more sensor(s) to sense one or more air quality parameter(s) from the air sample within the air sequestration chamber. The air quality analyzer also includes an analysis module to perform analysis of the sensed one or more air quality parameter(s) and to transmit data associated with the analysis over a communication link.

In yet another aspect, a method of a safety system of a structure having a fixed piping system implemented therewithin to facilitate delivery of breathable air from a source across the safety system is disclosed. The method includes segregating a portion of the breathable air for analysis as an air sample, and sensing one or more air quality parameter(s) from the air sample. The method also includes performing analysis of the sensed one or more air quality parameter(s), and transmitting data associated with the analysis over a communication link.

The methods and systems disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows

### BRIEF DESCRIPTION OF THE DRAWINGS

The embodiments of this invention are illustrated by way of example and not limitation in the figures of the accompanying drawings, in which like references indicate similar elements and in which:

FIG. 1 is a schematic view of a safety system of a structure, according to one or more embodiments.

FIG. 2 is a conceptual and an illustrative view of the safety system of FIG. 1 including a breathable air supply system, according to one or more embodiments.

FIG. 3 is an illustrative view of the air monitoring system of FIGS. 1-2, according to one or more embodiments.

FIG. 4 is an example schematic view of a display module of the air monitoring system or an air quality analyzer of the safety system of FIGS. 1-2.

FIG. 5 is an illustrative view of an example air quality analyzer of the safety system of FIGS. 1-2.

FIG. 6 is a schematic view of the air quality analyzer of the safety system of FIGS. 1-2, according to one or more embodiments.

FIG. 7 is a schematic view of example constituent sensors of the air quality analyzer of the safety system of FIGS. 1-2.

FIG. 8 is a schematic view of a communication context between the air quality analyzer, a data processing device and a remote certification laboratory of the safety system of FIGS. 1-2, according to one or more embodiments.

FIG. 9 is an example user interface view of an air safety application executing on the data processing device of the safety system of FIG. 2.

FIG. 10 is a process flow diagram detailing the operations involved in sensor-based breathable air quality monitoring in a safety system of a structure, according to one or more embodiments.

Other features of the present embodiments will be apparent from the accompanying drawings and from the detailed description that follows.

### DETAILED DESCRIPTION

Example embodiments, as described below, may be used to provide a method, a device and a system of sensor-based breathable air quality monitoring in a safety system of a structure. Although the present embodiments have been described with reference to specific example embodiments, it will be evident that various modifications and changes may be made to these embodiments without departing from the broader spirit and scope of the various embodiments.

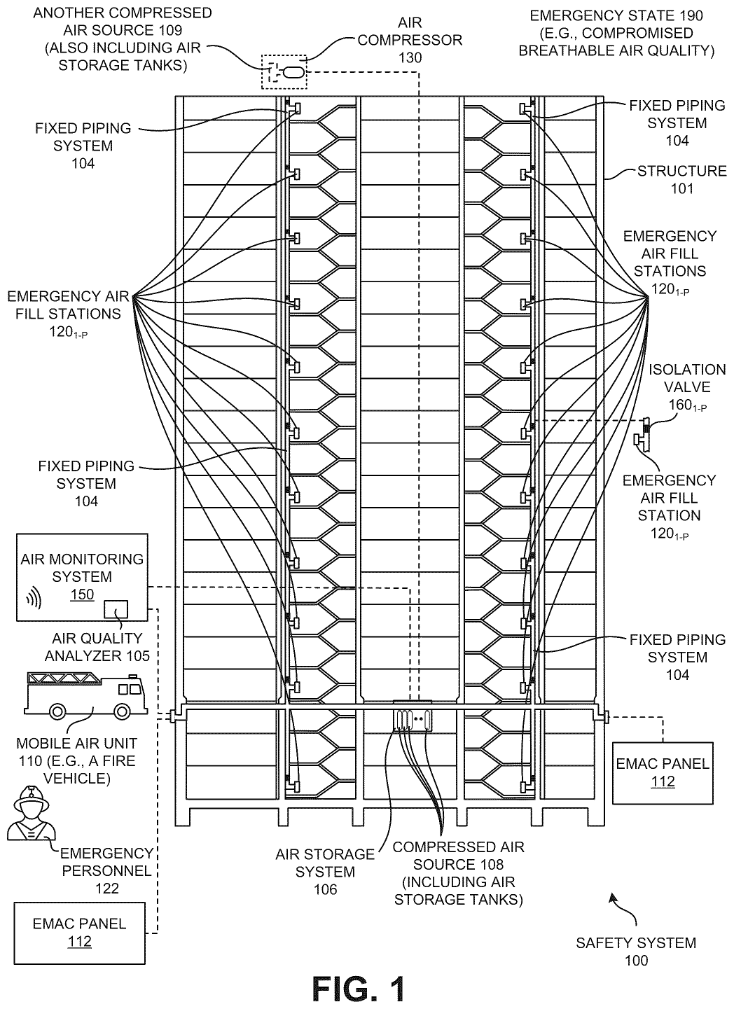

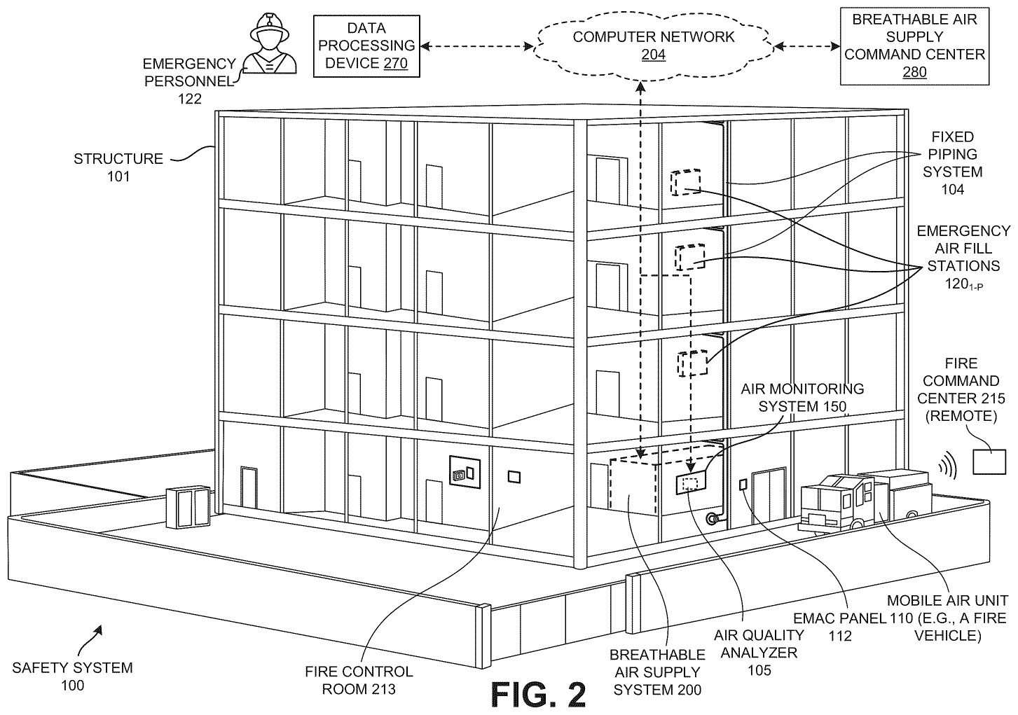

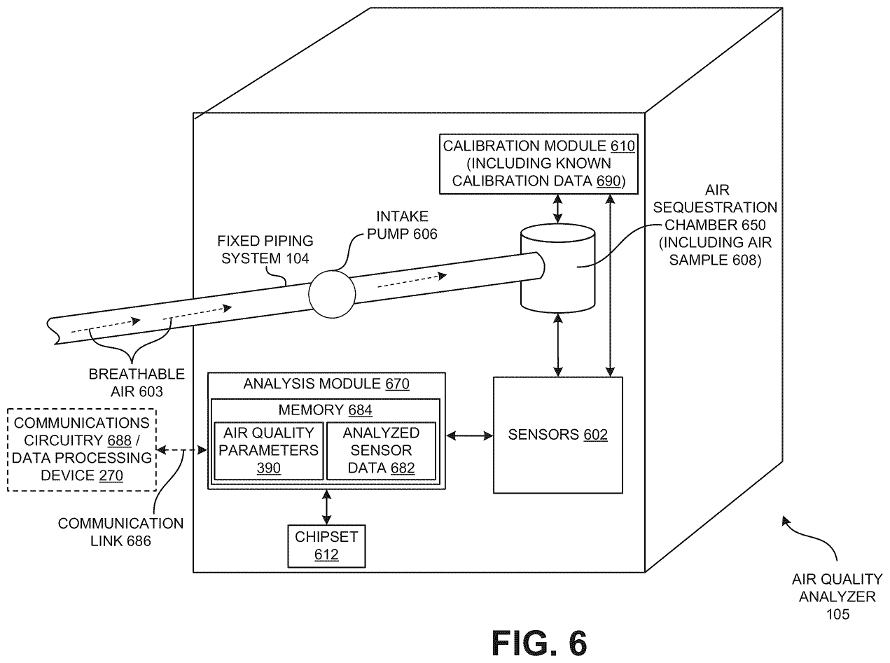

FIG. 1 shows a safety system 100 associated with a structure 101, according to one or more embodiments. In one or more embodiments, safety system 100 may be a Firefighter Air Replenishment System (FARS) to enable firefighters (e.g., emergency personnel 122) entering structure 101 in times of fire-related emergencies to gain access to breathable air (e.g., human breathable; refer to breathable air 603 in FIG. 6) in-house. In one or more embodiments, safety system 100 may supply the breathable air from an air storage system 106 stored in structure 101. In one or more embodiments, a breathable air supply system 200 (refer to FIG. 2) may facilitate delivery of the breathable air from a source (e.g., compressed air source 108) of compressed air through a fixed piping system 104 permanently installed within structure 101 serving as a constant source 108 of replenishment of the breathable air.

In one or more embodiments, structure 101 may encompass vertical building structures, horizontal building structures (e.g., shopping malls, hypermarts, extended shopping, storage and/or warehousing related structures), tunnels, marine craft (e.g., large marine vessels such as cruise ships, cargo ships, submarines and large naval craft, which may be “floating” versions of buildings and horizontal structures) and mines. Other structures are within the scope of the exemplary embodiments discussed herein.

In one or more embodiments, safety system 100 may include an air quality analyzer 105 coupled (e.g., permanently affixed) to fixed piping system 104. In one or more embodiments, air quality analyzer 105 may be configured to conduct a set of specialized tests to check and/or monitor the quality of breathable air within safety system 100. In one or more embodiments, air quality analyzer 105 may collect a sample (e.g., refer to air sample 608 of FIG. 6) of the breathable air from compressed air source 108 for analysis thereof before the breathable air is supplied within safety system 100.

As shown in FIG. 1, fixed piping system 104 may distribute air across floors/levels of structure 101. For the aforementioned purpose, fixed piping system 104 may distribute air from an air storage system 106 (e.g., within structure 101) including a number of air storage tanks (e.g., part of compressed air source 108, another compressed air source 109) that serves as a source of pressurized air (e.g., pressurized using air compressor 130 of another compressed air source 109). Additionally, in one or more embodiments, fixed piping system 104 may interconnect with a mobile air unit 110 (e.g., a fire vehicle) through an External Mobile Air Connection (EMAC) panel 112.

In one or more embodiments, an air monitoring system 150 may be installed as part of safety system 100 to automatically track and monitor a parameter (e.g., pressure) and/or a quality (e.g., indicated by moisture levels, carbon monoxide levels, etc.) of the breathable air within safety system 100. FIG. 1 shows air monitoring system 150 as communicatively coupled to air storage system 106 and EMAC panel 112 and including air quality analyzer 105. In one or more embodiments, for monitoring the parameters and/or the quality of the breathable air within safety system 100, air quality analyzer 105 may include appropriate sensors (to be discussed below) and circuitries therein. For example, a pressure sensor within air monitoring system 150 may automatically sense and record the pressure of the breathable air of safety system 100. Said pressure sensor may communicate with an alarm system that is triggered when the sensed pressure is outside a safe range. Also, in one or more embodiments, air monitoring system 150 may automatically trigger a shutdown of breathable air distribution through safety system 100 in case of impurity/contaminant (e.g., carbon monoxide) detection therethrough yielding levels above a safety threshold.

In one or more embodiments, fixed piping system 104 may include pipes (e.g., constituted out of stainless steel tubing) that distribute breathable air to a number of emergency air fill stations 120_{1-P} within structure 101. The emergency air fill station 120_{1-P} may be an emergency air fill panel and/or a rupture containment air fill station. In one or more embodiments, proximate each emergency air fill station 120_{1-P}, safety system 100 may include an isolation valve 160_{1-P} to isolate a corresponding emergency air fill station 120_{1-P} from a remainder of safety system 100. For example, said isolation may be achieved through the manual turning of isolation valve 160_{1-P} proximate the corresponding emergency air fill station 120_{1-P} and/or remotely from air monitoring system 150. It should be noted that configurations and components of safety system 100 may vary from the example safety system 100 of FIG. 1.

FIG. 2 shows a conceptual view of safety system 100 of FIG. 1 including breathable air supply system 200, according to one or more embodiments. In one or more embodiments, emergency personnel 122 may request an air quality test through air monitoring system 150 of breathable air supply system 200. In one or more embodiments, air quality analyzer 105 of air monitoring system 150 may automatically get activated (e.g., based on one or more control signals from data processing device 270 (e.g., a server, a network of distributed servers, a mobile device, a portable smart device) associated with emergency personnel 122 at a breathable air supply command center 280 (e.g., a remote fire command center 215, a fire control room 213 within structure 101) or otherwise) to collect an air sample of the breathable air from compressed air source 108 for analysis. In one or more embodiments, one or more sensors (to be discussed below) of air quality analyzer 105 may sense air quality parameters from the air sample. In one or more embodiments, air quality analyzer 105 may further transmit the sensed air quality parameters and/or data associated with an analysis thereof to a remote certification laboratory for an on-demand certification. In one or more embodiments, the remote certification laboratory may, in turn, perform the appropriate tests for certification and transmit the test results along with the certification for breathable air supply system 200 to emergency personnel 122 at data processing device 270.

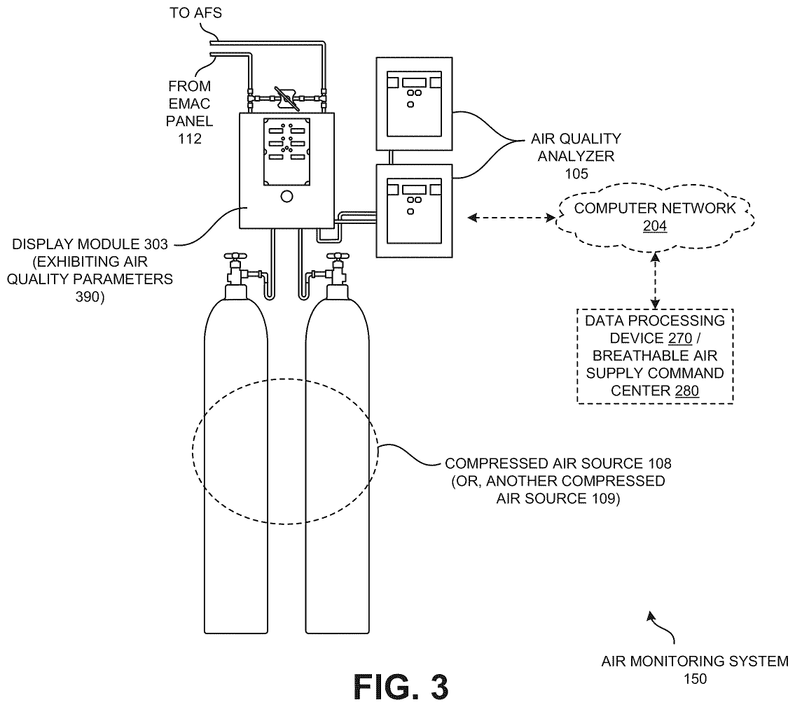

Thus, in one or more embodiments, components of safety system 100 including air quality analyzer 105/air monitoring system 150 may be communicatively coupled to data processing device 270/breathable air supply command center 280 via a computer network 204 (e.g., a Wide Area Network (WAN), a Local Area Network (LAN), a short-range communication network). FIG. 3 shows air monitoring system 150 of safety system 100 of FIGS. 1-2, according to one or more embodiments. In one or more embodiments, air monitoring system 150 may be a collection of units and/or components put together to check, monitor and record quality of the breathable air (e.g., breathable air 603) across safety system 100. In one or more embodiments, air monitoring system 150 may include a display module 303 to exhibit the air parameters (e.g., air quality parameters 390) captured and analyzed by air quality analyzer 105 of air monitoring system 150, e.g., in real-time. In one or more embodiments, display module 303 may be a display device (e.g., with a mini touchscreen) for a visual presentation of air quality parameters 390 and data analyzed by air quality analyzer 105.

In one or more embodiments, display module 303 may be installed at key locations of structure 101. In one or more implementations, display module 303 may be made of a material having fire-rated capabilities. In one or more embodiments, display module 303 may communicate through wired and/or wireless means (e.g., through computer network 204) with external devices including computing/data processing systems (e.g., data processing device 270). In some implementations, a camera (not shown) installed on or associated with display module 303 may be integrated with data processing device 270 to provide visual images and/or audio data in the vicinity thereof within structure 101. The air quality parameters may be monitored as per standard fire safety guidelines (e.g., National Fire Protection Association (NFPA), Occupational Safety and Health Administration (OSHA) and/or Compressed Gas Association (CGA) standards).

In one or more embodiments, air quality analyzer 105 may be a sensor-based device to automatically detect air quality parameters 390 on a continual basis. In an implementation, air monitoring system 150 may be equipped with not less than two content analyzers (e.g., air quality sensors) capable of detecting carbon monoxide, carbon dioxide, nitrogen, oxygen, moisture, and/or hydrocarbon levels in the breathable air. The sensors (to be discussed below) of air quality analyzer 105 may detect/sense deviation in air quality parameters 390 of the breathable air that is also displayed by the display module 303, according to one or more embodiments. In one or more embodiments, air quality analyzer 105 may be integrated with computer network 204; in one or more embodiments, in the case of computer network 204 being a cloud computing network, all capabilities and services offered therethrough may be leveraged by air monitoring system 150/air quality analyzer 105. In one or more embodiments, computer network 204 may store data from air quality analyzer 105 and/or data analyzed therefrom on the “cloud” (e.g., over the Internet).

In one or more embodiments, air quality analyzer 105 may continuously send air quality parameters 390 (e.g., air component parameters, temperature, pressure, etc.) of breathable air supply system 200 units to data processing device 270 (e.g., at breathable air supply command center 280) through computer network 204. In one or more embodiments, emergency personnel 122 at data processing device 270 may remotely manage and continuously monitor breathable air supply system 200 including air monitoring system 150/air quality analyzer 105.

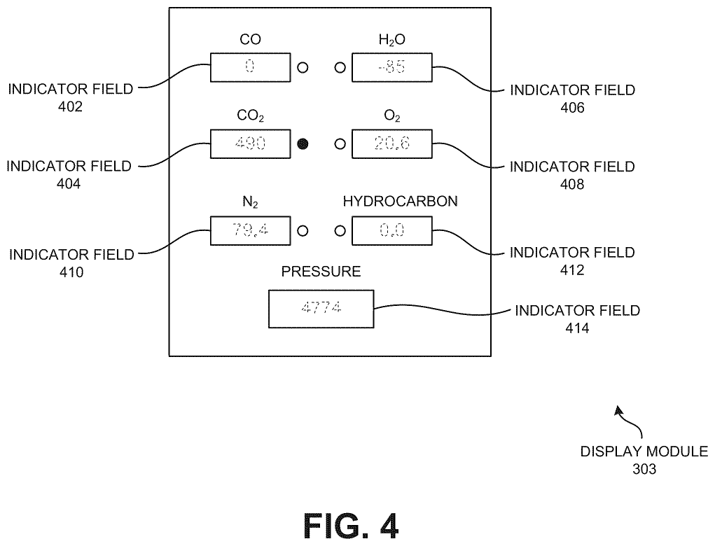

FIG. 4 shows an exemplary display module 303 of air monitoring system 150/air quality analyzer 105, according to one or more embodiments. In an example implementation indicated in FIG. 4, an indicator field 402 of display module 303 may display a carbon monoxide (CO) content in the breathable air from compressed air source 108 (or, another compressed air source 109), an indicator field 404 of display module 303 may display the carbon dioxide (CO₂) content in the breathable air, an indicator field 406 of display module 303 may display the moisture (H₂O) content in the breathable air, an indicator field 408 of display module 303 may display the oxygen (O₂) content in the breathable air, an indicator field 410 of display module 303 may display the nitrogen (N₂) content in the breathable air, and an indicator field 412 of display module 303 may display the hydrocarbon content in the breathable air. In addition, an indicator field 414 of display module 303 may display a pressure of the breathable air from compressed air source 108 (or, another compressed air source 109).

In one or more implementations, display module 303 may also include indicator lights (not shown; e.g., Light Emitting Diode (LED) based lights). For example, whenever certain air quality parameters 390 (e.g., temperature, pressure, moisture) go out of range, one or more indicator light(s) may change a color thereof from green to red, with the green color indicating that the relevant air quality parameters 390 are within threshold limits and the red light indicating that the aforementioned air quality parameters 390 are not within the threshold limits. Further, in some implementations, display module 303 may include a Quick Response (QR) code scanner (not shown) to enable authorized users (e.g., emergency personnel 122) to scan with data processing devices (e.g., data processing device 270 in the form of a mobile device) and check air quality parameters 390.

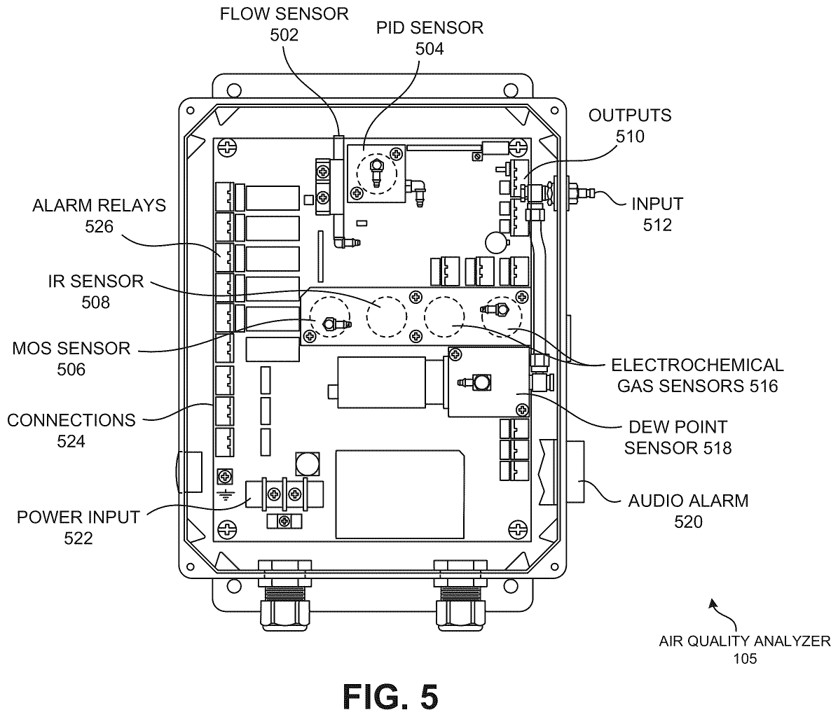

FIG. 5 shows an example air quality analyzer 105. Here, air quality analyzer 105 may include a flow sensor 502 (e.g., an electronic device) that measures and/or regulates a flow rate of the breathable air (e.g., from compressed air source 108, from another compressed air source 109) across safety system 100. Air quality analyzer 105 may also include a photoionization detector (PID) sensor 504 to detect a low concentration of volatile organic compounds (VOCs) within the breathable air. PID sensor 504 may detect/sense the presence of one or more hazardous substance(s) in the breathable air. PID sensor 504 may utilize an ultraviolet (UV) light source to break down the VOCs in the breathable air into positive and negative ions. PID sensor 504 may then detect and/or measure the charge of the ionized gas, where the charge is a function of the concentration of the VOCs in the breathable air.

A Metal Oxide Semiconductor (MOS) sensor 506 of air quality analyzer 105 may detect the concentration of various types of gases in the breathable air/air sample by measuring a change in resistance of a metal oxide due to adsorption of gases in the breathable air/air sample. An infrared (IR) sensor 508 of air quality analyzer 105 may measure and detect infrared radiation in a vicinity of air quality analyzer 105.

Outputs 510 may be in the form of electrical signals used to identify air components of the breathable air/air sample. The electrical signals may be generated by sensors including the sensors discussed herein. An input 512 may be an intake of the breathable air/air sample (e.g., through a hose) from compressed air source 108/another compressed air source 109/air storage system 106. An electromechanical gas sensor 516 of air quality analyzer 105 may be operated based on a diffusion of a gas of interest (e.g., air components of the breathable air/air sample) thereinto. Said diffusion may result in generation of an electrical signal proportional to a concentration of the gas of interest.

A dew point sensor 518 of air quality analyzer 105 may be used to measure and monitor a dew point temperature of the breathable air/air sample. An audio alarm 520 may be a transducer device to generate an audible alert once an emergency state is detected by air quality analyzer 105 based on data from the sensors. A power input 522 may be an input corresponding to an amount of energy put into and/or consumed by air quality analyzer 105. Connectors 524 maybe links between electrical components of air quality analyzer 105.

An alarm relay 526 may be an electric switch that activates circuitry to protect the sensors against abnormal power conditions. The abnormal power conditions may include but are not limited to voltage surges, electrical transients, short circuits, overvoltage and overcurrent. During said abnormal power conditions, alarm relay 526 may automatically isolate the sensors by opening and/or breaking the circuit from a power supply. Alarm relay 626 may also activate circuits/devices to bypass storage system 106/compressed air source 108/another compressed air source 109 when anomalies (e.g., contamination in the breathable air/air sample) and/or faults (e.g., fire hazards, pressure variations, deviation in air quality parameters 390) are detected by the sensors. In one or more embodiments, air monitoring system 150 may be made of fire-rated material to protect safety system 100 from physical damage during hazardous situations. Further, in one or more embodiments, air monitoring system 150 may be made of weather-resistant and/or UV/solar/infrared radiation-resistant material/material(s) to prevent corrosion and/or deterioration of components thereof due to prolonged exposure to harsh environmental and/or weather conditions.

FIG. 6 shows air quality analyzer 105 in detail, according to one or more embodiments. In one or more embodiments, as discussed above, air quality analyzer 105 may be a device that automatically detects air quality, moisture content, and/or pressure (e.g., all of the aforementioned may be part of air quality parameters 390) of the breathable air (e.g., breathable air 603) within safety system 100, for example, on a continual basis. In one or more embodiments, as air quality analyzer 105 requires a sample of breathable air 603 supplied through fixed piping system 104 for sensing air quality parameters 390 and for internal/external analyses thereof, air quality analyzer 105 may be coupled (e.g., permanently affixed) to fixed piping system 104.

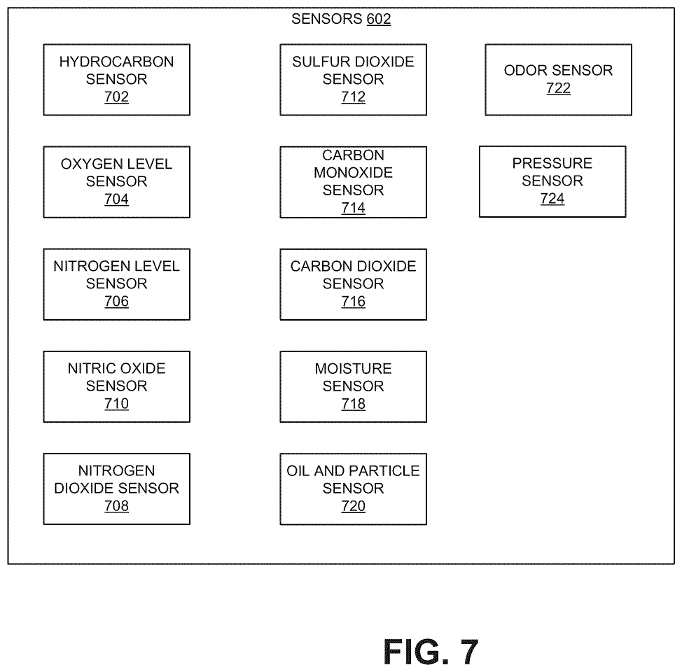

In one or more embodiments, an intake pump 606 may ingest a quantity of breathable air 603 through fixed piping system 104. In one or more embodiments, intake pump 606 may deliver the ingested breathable air 603 into an air sequestration chamber 650 inside air quality analyzer 105. In one or more embodiments air sequestration chamber 650 may segregate a portion of breathable air 603 for analysis as an air sample 608. In one or more embodiments, air sequestration chamber 650 may be communicatively coupled with sensors 602 (e.g., an array of sensors including but not limited to flow sensor 502, PID sensor 504, MOS sensor 506 and the sensors to be discussed in conjunction with FIG. 7). In one or more embodiments, sensors 602 may detect air quality parameters 390 from air sample 608 within air sequestration chamber 650. In some embodiments, breathable air 603 from fixed piping system 104 may directly fill air sequestration chamber 650 instead of intake pump 606 being utilized therefor.

In one or more embodiments, an analysis module 670 of air quality analyzer 105 may receive the sensed (e.g., by sensors 602) air quality parameters 390 and perform analyses (e.g., analyzed sensor data 682) thereof. FIG. 6 shows analysis module 670 as including a memory 684 with air quality parameters 390 and analyzed sensor data 682 stored therein. In one or more embodiments, analysis module 670 may also be associated with a chipset 612 coupled thereto to convert air quality parameters 390 (or, any data associated with air quality parameters 390) into computer-readable forms thereof. In one or more embodiments, analyzed sensor data 682 (and air quality parameters 390) may be transmitted by analysis module 670 over a communication link 686 (e.g., representing electrical communication to communications circuitry 688 to transmit results of the analyses; communications circuitry 688 may be associated with a Graphical User Interface (GUI) or a plug-in readout module for local reading; said communications circuitry 688 may also be associated with display module 303 discussed above).

In one or more embodiments, communication link 686 may also represent a communication interface to computer network 204 whereby air quality parameters 390 and/or analyzed sensor data 682 are transmitted by analysis module 670 to data processing device 270 (e.g., remote from air quality analyzer 105) for analyses/further analyses thereat. In the case of analysis module 670 being internal to air quality analyzer 105 as in FIG. 6, analysis module 670 may also perform a set of predefined tests on air sample 608 using air quality parameters 390. Here, in one or more embodiments, communication link 686 may transmit the test results to data processing device 270 over computer network 204. In one or more embodiments, as seen with reference to FIG. 3, air quality analyzer 105 may include or at least be associated with display module 303 to display air quality parameters 390 and/or analyzed sensor data 682.

In one or more embodiments, air sample 608 may be released (e.g., using an electronically controlled valve) following completion of analysis of air quality parameters 390 by analysis module 670 after which a new air sample (e.g., analogous to air sample 608) may be ingested (or supplied) into air sequestration chamber 650. In one or more embodiments, air quality analyzer 105 may further include a calibration module 610 (e.g., a device/device module) associated with sensors 602 to compare a characteristic (e.g., a zero error) of breathable air 603/air sample 608 based on analyzed sensor data 682 to known calibration data 690 stored in calibration module 610. In one or more embodiments, response to determining that the characteristic is dissimilar to the known calibration data 690, calibration module 610 may adjust parameters of air quality analyzer 105 to account for the dissimilarity.

In one or more embodiments, air quality analyzer 105 may, via communication link 686, receive instructions from breathable air supply command center 280 (e.g., remote fire command center 215, fire control room 213) to transform and/or transition safety system 100 to an emergency state (e.g., emergency state 190 in FIG. 1). In accordance therewith, in one or more embodiments, breathable air supply command center 280 (e.g., through data processing device 270) may direct safety system 100/breathable air supply system 200 to acquire breathable air 603 from a different source (e.g., another compressed air source 109) instead of a current source (e.g., compressed air source 108) when detection of breathable air 603/air sample 608 through air quality analyzer 105 indicates a compromised quality (e.g., based on one or more air quality parameters 390 exceeding predefined thresholds) of breathable air 603.

In one or more embodiments, as discussed above, air quality analyzer 105 may transmit air quality parameters 390 and/or analyzed sensor data 682 over communication link 686 (e.g., via computer network 204) to a remote certification laboratory (e.g., can also be regarded as being hosted on data processing device 270). In one or more embodiments, the remote certification laboratory may employ a set of certified and/or accredited professionals (e.g., can also be regarded as emergency personnel 122) who remotely certify the FARS of structure 101 based on the received air quality parameters 390 and/or analyzed sensor data 682.

In one or more embodiments, the remote certification laboratory may manually and/or automatically conduct a set of specialized tests through air quality analyzer 105, such as longer tests (e.g., 30 minutes or more) and shorter tests (e.g., just a few minutes) on demand. For the aforementioned purpose, in one or more embodiments, the remote certification laboratory may transmit appropriate control signals. Thus, in one or more embodiments, the remote certification laboratory may have full access to monitor, control, regulate, and operate safety system 100/breathable air supply system 200 during certification of safety system 100. It should be noted that other components (e.g., emergency air fill stations 120_{1-P}) of safety system 100 may have sensors analogous to sensors 602 and devices analogous to air quality analyzer 105. In one or more embodiments, remote control and certification of one or more components of safety system 100 may thus be possible based on access to data analogous to air quality parameters 390 and/or analyzed sensor data 682 via communication links analogous to communication link 686. In some embodiments, each time safety system 100 is certified, a certification may be written permanently into a distributed ledger and/or a blockchain (e.g., Ethereum™ blockchain, Solana™ blockchain) associated with data processing device 270 for redundant and/or secondary record keeping purposes.

FIG. 7 shows example constituent sensors of sensors 602 of air quality analyzer 105. The example constituent sensors of sensors 602 may include but are not limited to a hydrocarbon sensor 702, an oxygen level sensor 704, a nitrogen level sensor 706, a nitrogen dioxide sensor 708, a nitric oxide sensor 710, a sulfur dioxide sensor 712, a carbon monoxide sensor 714, a carbon dioxide sensor 716, a moisture sensor 718, an oil and particle sensor 720, an odor sensor 722 and a pressure sensor 724. In one or more embodiments, air quality analyzer 105 may receive instructions from breathable air supply command center 280 (e.g., at data processing device 270) to automatically transition and/or transform safety system 100 to emergency state 190 when one or more of the following conditions occur:
1. Carbon monoxide sensor 714 senses a level of carbon monoxide in breathable air 603/air sample 608 to be above a first predetermined threshold (e.g., 4.5 parts per million (ppm),
2. Carbon dioxide sensor 716 senses a level of carbon dioxide in breathable air 603/air sample 608 in excess of a second predetermined threshold (e.g., 1,000 ppm),
3. Oxygen level sensor 704 senses that a level of oxygen in breathable air 603/air sample 608 falls outside a third predetermined threshold (e.g., a range of between 19.5% and 23.5%),
4. Nitrogen level sensor 706 senses that a level of nitrogen in breathable air 603/air sample 608 falls below a fourth predetermined threshold (e.g., 75%) or rises above a fifth predetermined threshold. (e.g., 81%),
5. Hydrocarbon sensor 702 senses that a hydrocarbon content in breathable air 603/air sample 608 exceeds a sixth predetermined threshold (e.g., 5 milligrams per cubic meter of air),
6. Moisture sensor 718 senses that a moisture concentration in breathable air 603/air sample 608 exceeds a seventh predetermined threshold (e.g., 24 ppm by volume),
7. Pressure sensor 724 senses that a pressure level of breathable air 603/air sample 608 falls below an eighth predetermined threshold (e.g., 90% of a maintenance pressure specified in a fire code),
8. Nitric oxide sensor 710 senses a nitric oxide concentration in breathable air 603/air sample 608 in excess of a safety level prescribed as per the standard fire safety guidelines discussed above,
9. Oil and particle sensor 720 senses an oil and particle concentration in breathable air 603/air sample 608 in excess of another safety level prescribed as per the standard fire safety guidelines discussed above.
10. Sulfur dioxide sensor 712 senses a sulfur dioxide concentration in breathable air 603/air sample 608 outside a threshold safety range as per the standard fire safety guidelines discussed above, and
11. Odor sensor 722 senses an odor level in breathable air 603/air sample 608 outside yet another safety level prescribed in the standard fire safety guidelines discussed above.

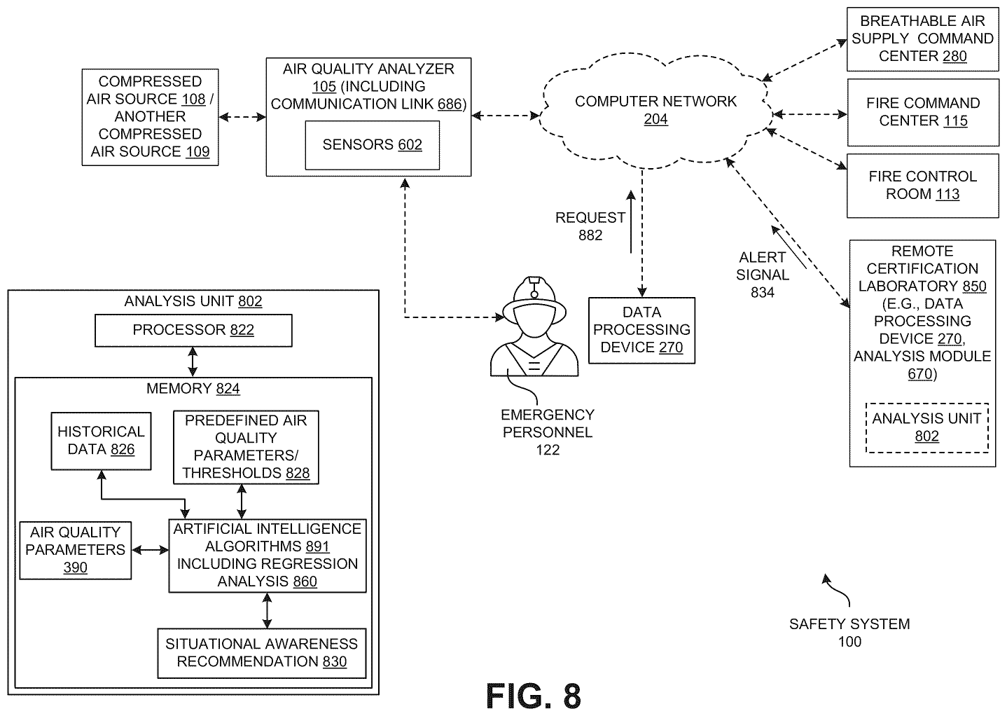

In some embodiments, air quality analyzer 105/air monitoring system 150 may be configured to automatically detect emergency state 190 in accordance with the conditions discussed above. FIG. 8 shows a communication context between air quality analyzer 105, data processing device 270 and a remote certification laboratory 850 (e.g., at data processing device 270), according to one or more embodiments. In one or more embodiments, emergency personnel 122 at data processing device 270 (e.g., a mobile device, at remote certification laboratory 850) may request (e.g., request 882 transmitted) a test of air quality analyzer 105 for an on-demand air quality certification thereof. In one or more embodiments, request 882 (e.g., received at air quality analyzer 105 via communication link 686) in the form of a control signal may activate air quality analyzer 105 for collection of air quality parameters 390 and/or analyzed sensor data 682.

In one or more embodiments, reception of air quality parameters 390 and/or analyzed sensor data 682 via computer network 204 at remote certification laboratory 850 may render it feasible for the remote certification of safety system 100/air monitoring system 150/air quality analyzer 105 to occur. In one or more embodiments, remote certification laboratory 850 may include an analysis unit 802 (e.g., a data processing device such as a server) including a processor 822 (e.g., a processor core, a network of processors, a processor) communicatively coupled to a memory 824 (e.g., a volatile and/or a non-volatile memory and/or a database). In one or more embodiments, memory 824 may have historical data 826 (e.g., relevant to safety system 100/air quality analyzer 105 and breathable air 603 therein) and predefined air quality parameters/thresholds 828 (e.g., nitrogen level, oxygen level, carbon monoxide level, pressure level) relevant to breathable air 603 discussed above stored therein.

In some embodiments, analysis module 670 may also be at data processing device 270/remote certification laboratory 850; here, data processing device 270/remote certification laboratory 850 may receive air quality parameters 390 via computer network 204 and perform analysis thereof. In one or more embodiments, analysis unit 802 may utilize air quality parameters 390 and predefined air quality parameters/thresholds 828 to automatic bypass air storage system 106/compressed air source 108/another compressed air source 109 discussed above. In some embodiments, analysis unit 802 may execute one or more artificial intelligence algorithms 891 (e.g., stored in memory 824 and executable through processor 822) for advanced profiling and/or testing of breathable air 603/air sample 608 through safety system 100. In one or more embodiments, in accordance with analysis module 670 being external to air quality analyzer 105, a situational awareness recommendation 830 (e.g., reaction protocols during emergency state 190) may be transmitted to breathable air supply command center 280 and/or data processing device 270 (e.g., associated with emergency personnel 122) through/via/over communication link 686; the one or more artificial intelligence algorithms 891 may be employed for regression analysis 860 of air quality parameters 390 to provide situational awareness recommendation 830.

In some embodiments, the profiling and/or testing through analysis unit 802 of remote certification laboratory 850 may provide for accreditation of air quality of breathable air 603 within safety system 100 when the results of the profiling/testing yield that air quality parameters 390 are within the predefined air quality parameters/thresholds 828; the aforementioned accreditation may be provided in the form of a certificate to breathable air supply command center 280 and/or data processing device 270 (e.g., associated with emergency personnel 122). In one or more embodiments, when the results of the profiling/testing yield that air quality parameters 390 are not within predefined air quality parameters/thresholds 828, remote certification laboratory 850/analysis unit 802 may generate alert signal 834 to notify breathable air supply command center 280 and/or data processing device 270 of emergency state 190 of safety system 100.

In some implementations, alert signal 834 may automatically activate appropriate devices to switch off supply of breathable air 603 from compressed air source 108/another compressed air source 109/air storage system 106 and, thereby, isolate compressed air source 108/another compressed air source 109/air storage system 106 from safety system 100. Alert signal 834 additionally may activate the appropriate devices to automatically connect a different compressed air source (e.g., another compressed air source 109) to safety system 100/emergency air fill stations 120_{1-P} to ensure a continuous supply of breathable air 603 within safety system 100, according to one or more embodiments.

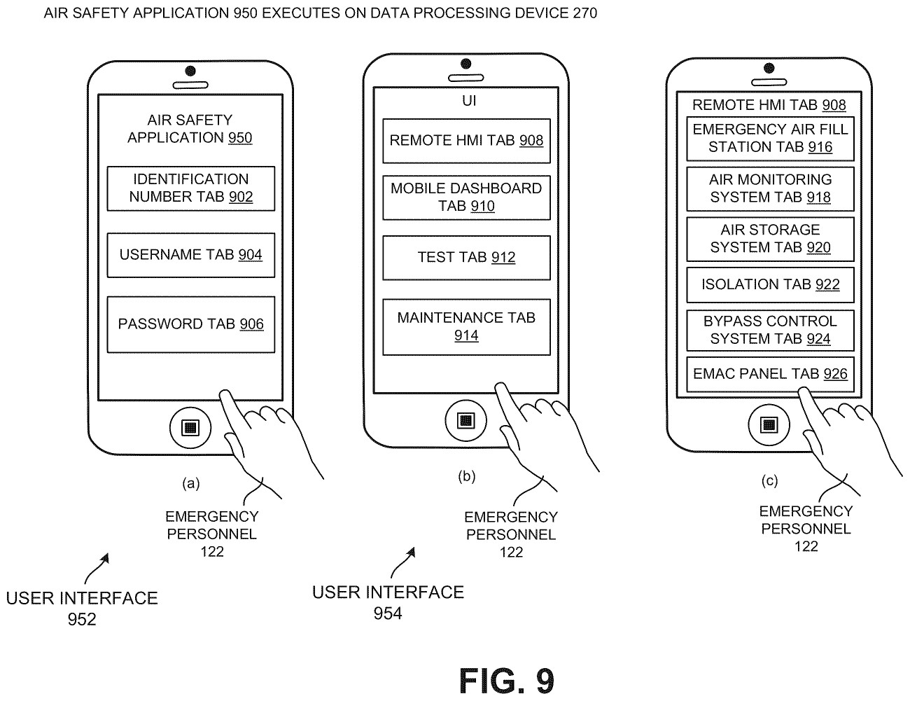

FIG. 9 shows an example user interface 952 of an air safety application 950 executing on data processing device 270 (e.g., on a processor communicatively coupled to a memory thereof; data processing device 270 may be a mobile device or a server; data processing device 270 may even be remote certification laboratory 850, in which case air safety application 950 may execute on processor 822). As shown in ‘(a)’, user interface 952 may display user authentication tabs of air safety application 950. Example user authentication tabs may include an identification number tab 902, a username tab 904, and a password tab 906. Emergency personnel 122 (e.g., authorized users, firefighters, emergency responses) may need to enter a corresponding identification number, username and password to access features provided through air safety application 950.

As shown in ‘(b)’, upon authentication, example user interface 954 may display a remote Human-Machine Interface (HMI) tab 908, a mobile dashboard tab 910, a test tab 912, and a maintenance tab 914. Remote HMI tab 908 may help emergency personnel 122 to remotely control safety system 100. Mobile dashboard tab 910 may help show a real-time graphical display of an entirety of safety system 100. Test tab 912 may help emergency personnel 122 to request analysis of breathable air 603 through remote certification laboratory 850 and generate custom reports. Maintenance tab 914 may help provide a proactive dimension to view upcoming and/or current maintenance requirements of safety system 100.

As shown in ‘(c)’, remote HMI tab 908 may display an emergency air fill station tab 916, an air monitoring system tab 918, an air storage system tab 920, an isolation tab 922, a bypass control system tab 924, and an EMAC panel tab 926. Remote HMI tab 908 may enable emergency personnel 122 to control components associated with the aforementioned tabs to effect an automatic bypass of air storage system 106/compressed air source 108/another compressed air source 109, as discussed above, and obtain air quality parameters 390. Based on zeroing in on specific tabs discussed herein, more detailed operations such as controlling relay devices, requesting certification through remote certification laboratory 850, purging breathable air 603 from safety system 100, isolating compressed air source 108/another compressed air source 109/air storage system 106 and so on are within the scope of the exemplary embodiments discussed herein.

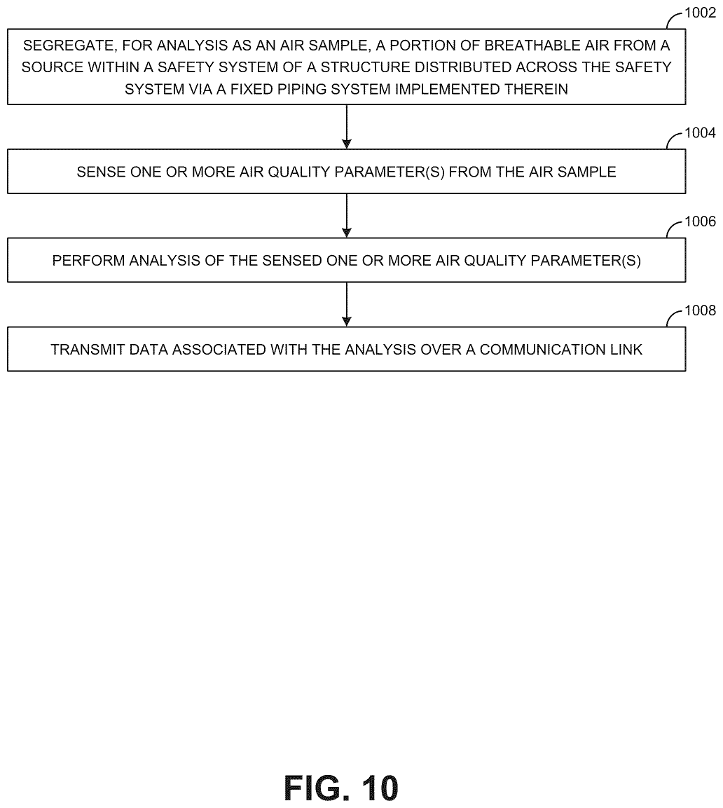

FIG. 10 shows a process flow diagram detailing the operations involved in sensor-based breathable air quality monitoring in a safety system (e.g., safety system 100) of a structure (e.g., structure 101) having a fixed piping system (e.g., fixed piping system 104) implemented therewithin to facilitate delivery of breathable air (e.g., breathable air 603) from a source (e.g., compressed air source 108, another compressed air source 109) across the safety system, according to one or more embodiments. In one or more embodiments, operation 1002 may involve segregating (e.g., in an air sequestration chamber 650 inside air quality analyzer 105) a portion of the breathable air for analysis as an air sample (e.g., air sample 608). In one or more embodiments, operation 1004 may involve sensing one or more air quality parameter(s) (e.g., air quality parameters 390) from the air sample. In one or more embodiments, operation 1006 may involve performing analysis (e.g., through analysis module 670 internal or external to air quality analyzer 105) of the sensed one or more air quality parameter(s). In one or more embodiments, operation 1008 may then involve transmitting data associated with the analysis over a communication link (e.g., communication link 686).

Although the present embodiments have been described with reference to specific example embodiments, it will be evident that various modifications and changes may be made to these embodiments without departing from the broader spirit and scope of the various embodiments.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1. A safety system of a structure, comprising:
a fixed piping system installed within the structure to facilitate delivery of breathable air from a source of compressed air;
an air quality analyzer coupled to the fixed piping system, comprising:
an air sequestration chamber in which a portion of the breathable air is segregated for analysis as an air sample; and
at least one sensor to sense at least one air quality parameter from the air sample within the air sequestration chamber; and
an analysis module to perform analysis of the sensed at least one air quality parameter based on coupling thereof to the air quality analyzer and to transmit data associated with the analysis over a communication link.

2. The safety system of claim 1, wherein the air sample is released from the air sequestration chamber following the analysis of the sensed at least one air quality parameter through the analysis module. and a new air sample from the breathable air is received within the air sequestration chamber.

3. The safety system of claim 1, wherein the air quality analyzer further comprises:
a calibration module associated with the at least one sensor to: compare a characteristic of the air sample based on the analysis of the sensed at least one air quality parameter to known calibration data stored in the calibration module, and, in response to determining that the characteristic is dissimilar to the known calibration data, to adjust the air quality analyzer to account for the determined dissimilarity.

4. The safety system of claim 1, wherein the air quality analyzer further comprises a display module to display a result associated with the sensing of the at least one air quality parameter through the at least one sensor.

5. The safety system of claim 1, wherein the air quality analyzer further comprises a chipset to convert data associated with the sensed at least one air quality parameter into a computer-readable form.

6. The safety system of claim 1, wherein at least one of:
the air quality analyzer is permanently affixed to the fixed piping system,
the air quality analyzer further comprises an intake pump to ingest the portion of the breathable air,
the analysis module is at least one of: a remote data processing device coupled to the air quality analyzer through a computer network and internal to the air quality analyzer, and
the communication link represents a communication interface to the computer network.

7. The safety system of claim 1, wherein the at least one sensor comprises at least one of: a hydrocarbon sensor, an oxygen sensor, a nitrogen sensor, a nitrogen dioxide sensor, a nitric oxide sensor, a sulfur dioxide sensor, a carbon monoxide sensor, a carbon dioxide sensor, a moisture sensor, an oil and particle sensor, an odor sensor and a pressure sensor.

8. The safety system of claim 1, wherein the communication link receives instructions from at least one of: a fire command center and a fire control room:
to transform the safety system to an emergency state, and
to direct the safety system to acquire the breathable air from a different source when the sensed at least one air quality parameter is determined to be associated with a compromised quality of the breathable air.

9. The safety system of claim 8, wherein the emergency state is triggered when at least one of:
a carbon monoxide sensor of the at least one sensor senses that a level of carbon monoxide in the air sample exceeds a first predetermined threshold,
a carbon dioxide sensor of the at least one sensor senses that a level of carbon dioxide in the air sample exceeds a second predetermined threshold,
an oxygen level sensor of the at least one sensor senses that a level of oxygen in the air sample falls outside a third predetermined threshold,
a nitrogen level sensor of the at least one sensor senses that a level of nitrogen in the air sample one of: falls below a fourth predetermined threshold and rises above a fifth predetermined threshold,
a hydrocarbon sensor of the at least one sensor senses that a hydrocarbon content in the air sample exceeds a sixth predetermined threshold,
a moisture sensor of the at least one sensor senses that a moisture concentration in the air sample exceeds a seventh predetermined threshold, and
a pressure sensor of the at least one sensor senses that a pressure level of the air sample falls below an eighth predetermined threshold.

10. The safety system of claim 8, wherein a situational awareness recommendation is automatically transmitted through the communication link to the at least one of: the fire command center and the fire control room.

11. The safety system of claim 10, wherein the situational awareness recommendation is provided in accordance with execution of an artificial intelligence algorithm based on a regression analysis involving the at least one air quality parameter.

12. An air quality analyzer coupled to a fixed piping system implemented within a safety system of a structure, the fixed piping system distributing breathable air from a source across the safety system, the air quality analyzer comprising:
an air sequestration chamber in which a portion of the breathable air is segregated for analysis as an air sample;
at least one sensor to sense at least one air quality parameter from the air sample within the air sequestration chamber; and
an analysis module to perform analysis of the sensed at least one air quality parameter and to transmit data associated with the analysis over a communication link.

13. The air quality analyzer of claim 12, wherein at least one of:
the air sample is released from the air sequestration chamber following the analysis of the sensed at least one air quality parameter through the analysis module and a new air sample from the breathable air is segregated for subsequent analysis,
the air quality analyzer further comprises a calibration module associated with the at least one sensor to: compare a characteristic of the air sample based on the analysis of the sensed at least one air quality parameter to known calibration data stored in the calibration module, and, in response to determining that the characteristic is dissimilar to the known calibration data, to adjust the air quality analyzer to account for the determined dissimilarity, and
the air quality analyzer further comprises an intake pump to ingest the portion of the breathable air.

14. The air quality analyzer of claim 12, further comprising at least one of:
a display module to display a result associated with the sensing of the at least one air quality parameter through the at least one sensor, and
a chipset to convert data associated with the sensed at least one air quality parameter into a computer-readable form.

15. A method of a safety system of a structure having a fixed piping system implemented therewithin to facilitate delivery of breathable air from a source across the safety system, comprising:
segregating a portion of the breathable air for analysis as an air sample;
sensing at least one air quality parameter from the air sample;
performing analysis of the sensed at least one air quality parameter; and
transmitting data associated with the analysis over a communication link.

16. The method of claim 15, further comprising:
releasing the air sample following the analysis of the sensed at least one air quality parameter; and
ingesting a new air sample from the breathable air for subsequent analysis.

17. The method of claim 15, further comprising:
comparing a characteristic of the air sample based on the analysis of the sensed at least one air quality parameter to known calibration data; and
in response to determining that the characteristic is dissimilar to the known calibration data, adjusting the air quality analyzer to account for the dissimilarity.

18. The method of claim 15, comprising at least one of:
ingesting the portion of the breathable air using an intake pump;
segregating the portion of the breathable air as the air sample in an air sequestration chamber;
sensing the at least one air quality parameter from the air sample using at least one sensor; and
performing the analysis of the sensed at least one air quality parameter using an analysis module associated with the at least one sensor.

19. The method of claim 18, further comprising at least one of:
the air quality analyzer being permanently affixed to the fixed piping system;
the analysis module being at least one of: a remote data processing device coupled to the air quality analyzer through a computer network and internal to the air quality analyzer; and
the communication link representing a communication interface to the computer network.

20. The method of claim 15, further comprising receiving via the communication link instructions from at least one of: a fire command center and a fire control room:
to transform the safety system to an emergency state, and
to direct the safety system to acquire the breathable air from a different source when the sensed at least one air quality parameter is determined to be associated with a compromised quality of the breathable air.
